SEQUENCE LISTING (19 sequences)

SEQ ID NO: 1 - protein, 25 aa, synthetic construct
IQAASRIIRNEQFAIRRFQSMPVRL

SEQ ID NO: 2 - protein, 18 aa, synthetic construct
YILTHFKGFWNVVNNIPF

SEQ ID NO: 3 - protein, 20 aa, synthetic construct
EVFGLVPGLMMYATIWLREH

SEQ ID NO: 4 - protein, 16 aa, synthetic construct
WMALESILHRIYTHQS

SEQ ID NO: 5 - protein, 15 aa, synthetic construct
RELIIEFSKMARDPQ

SEQ ID NO: 6 - protein, 18 aa, synthetic construct
LKIFPSKRILRRHKRDWV

SEQ ID NO: 7 - protein, 20 aa, synthetic construct
RDTKIFYSITGPGADSPPEG

SEQ ID NO: 8 - protein, 16 aa, synthetic construct
YELAHQLPLPHNVSSH

SEQ ID NO: 9 - protein, 23 aa, synthetic construct
MRLTISYLRVRKLLDAGDLDIED

SEQ ID NO: 10 - protein, 25 aa, synthetic construct
LKALDGFVMVLTDDGDMIYISDNVN

SEQ ID NO: 11 - protein, 41 aa, synthetic construct
AEQTFEQAIQAASRIIRNEQFAIRRFQSMPVRLLGHSPVLR

SEQ ID NO: 12 - protein, 47 aa, synthetic construct
NTVHYILTHFKGFWNVVNNIPFLRNAGQEVFGLVPGLMMYATIWLRE

SEQ ID NO: 13 - protein, 46 aa, synthetic construct
PIKWMALESILHRIYTHQSDVWSYGKFRELIIEFSKMARDPQRYLV

SEQ ID NO: 14 - protein, 72 aa, synthetic construct
LKIFPSKRILRRHKRDWVVAPISVPENGKGPFPQRLNQLKSNKDRDTKIFYSITGPGADSPPEGVFAVEKET

SEQ ID NO: 15 - protein, 90 aa, synthetic construct
RSKESEVFYELAHQLPLPHNVSSHLDKASVMRLTISYLRVRKLLDAGDLDIEDDMKAQMNCFYLKALDGFVMVLTDDGDMIYISDNVNKY

SEQ ID NO: 16 - protein, 6 aa, synthetic construct
GGGGGS

SEQ ID NO: 17 - protein, 5 aa, synthetic construct
GPGPG

SEQ ID NO: 18 - protein, 321 aa, synthetic construct
MAEQTFEQAIQAASRIIRNEQFAIRRFQSMPVRLLGHSPVLRGGGGGSNTVHYILTHFKGFWNVVNNIPFLRNAGQEVFGLVPGLMMYATIWLREGGGGGSRSKESEVFYELAHQLPLPHNVSSHLDKASVMRLTISYLRVRKLLDAGDLDIEDDMKAQMNCFYLKALDGFVMVLTDDGDMIYISDNVNKYGGGGGSLKIFPSKRILRRHKRDWVVAPISVPENGKGPFPQRLNQLKSNKDRDTKIFYSITGPGADSPPEGVFAVEKETGGGGGSPIKWMALESILHRIYTHQSDVWSYGKFRELIIEFSKMARDPQRYLV

SEQ ID NO: 19 - DNA, 963 bp, synthetic construct
  source 1..963: mol_type = other DNA
atggccgagcagaccttcgagcaggccatccaggccgccagcaggatcatccgcaacgagcagttcgccatccggagatttcagagcatgccagtgaggctgctgggacactcccccgtgctgaggggaggaggaggaggctccaataccgtgcactacatcctgacacacttcaagggcttttggaacgtggtgaacaatatccctttcctgcggaatgccggccaggaggtgtttggcctggtgccaggcctgatgatgtacgccacaatctggctgagggagggaggaggaggaggatctagaagcaaggagtctgaggtgttttatgagctggcacaccagctgccactgccacacaacgtgagctcccacctggacaaggcctctgtgatgcggctgaccatcagctacctgcgggtgagaaagctgctggatgccggcgacctggacatcgaggacgatatgaaggcccagatgaattgcttctatctgaaggccctggacggctttgtgatggtgctgacagacgatggcgacatgatctacatctccgataacgtgaataagtatggcggcggcggcggcagcctgaagattttcccatccaagaggatcctgaggcgccacaagagggactgggtggtggcacccatctccgtgcctgagaacggcaagggcccttttccacagcggctgaaccagctgaagtctaataaggacagagataccaagattttctactccatcacaggaccaggagcagattctccacctgagggcgtgtttgcagtggagaaggagactggaggaggaggaggctctcctatcaagtggatggccctggagagcatcctgcacaggatctacacacaccagtccgacgtgtggtcttatggcaagttccgcgagctgatcatcgagtttagcaagatggcccgggatcctcagagatacctggtg

(19) United States
(12) Patent Application Publication
(10) Pub. No.: US 2026/0263576 A1
(43) Pub. Date: Sep. 10, 2026

(54) DNA VACCINE TARGETING OVEREXPRESSED ANTIGENS IN COLORECTAL CANCER

(71) Applicant: UNIVERSITY OF WASHINGTON, SEATTLE, WA (US)

(72) Inventors: Mary L. Disis, SEATTLE, WA (US); Denise Cecil, SEATTLE, WA (US)

(73) Assignee: UNIVERSITY OF WASHINGTON, SEATTLE, WA (US)

(21) Appl. No.: 19/165,403

(22) PCT Filed: Mar. 13, 2024
(86) PCT No.: PCT/US2024/019713

§ 371 Date: Sep. 15, 2025

Related U.S. Application Data
(60) Provisional application No. 63/490,881, filed on Mar. 17, 2023.

(51) Int. Cl. A61K 39/00; A61P 37/04; C07K 14/005; C07K 14/47; C07K 14/705; C07K 14/71; C12N 9/00; C12N 9/16

(52) U.S. Cl. CPC A61K 39/001154; A61K 39/001102; A61K 39/001104; A61K 39/001152; A61P 37/04; C07K 14/005; C07K 14/47; C07K 14/705; C07K 14/71; C12N 9/00; C12N 9/16; C12Y 301/03048; A61K 2039/53; A61K 2039/575; C07K 2319/00

(57) ABSTRACT

A vaccine in the form of a five-antigen poly-epitope DNA plasmid and related molecules designed to optimize anti-genicity, immunogenicity and physicochemical properties of the encoded fusion protein. All proteins in the construct were confirmed to be expressed and vaccination generated high magnitude IFN-γ in the majority of animals evaluated. No toxicities attributable to vaccination were observed. Anti-tumor efficacy of vaccination is confirmed in treated mice. The epitopes include certain portions of CDC25B; COX2; HIF-1alpha; CDH3; and EGFR.

This application claims benefit of U.S. provisional patent application No. 63/490,881, filed Mar. 17, 2023, the entire contents of which are incorporated by reference into this application.

### ACKNOWLEDGEMENT OF GOVERNMENT SUPPORT

This invention was made with government support under Grant No. 75N91019D00019; Task order 75N91019F00131, awarded by the National Institutes of Health. The government has certain rights in the invention.

### REFERENCE TO A SEQUENCE LISTING

The content of the XML file of the sequence listing named “UW81_seq”, which is 19 kb in size, created on Mar. 6, 2024, and electronically submitted herewith the application, is incorporated herein by reference in its entirety.

### BACKGROUND

Colorectal cancer (CRC) is the third most common cancer worldwide. About 1.2 million people will be diagnosed with CRC this year and 600,000 individuals will succumb to the disease. If a vaccine to prevent CRC could be developed to have the same protective efficacy as the human papilloma virus vaccine more than half a million lives could be saved each year. A vaccine approach is well suited to CRC prevention as a limited number of immunizations can be given over a short period of time to achieve a desired immune response. Further, if T-cells are appropriately activated by a vaccine the cells can traffic to antigen aberrantly expressed in neoplastic tissue anywhere in the body. Most importantly if an immunologic memory response develops, antigen specific T-cells will persist in the body for years ready to be deployed when aberrant cells are detected.

Active immunization as a cancer immuno-prevention strategy offers several advantages to classic drug-based approaches. First, vaccines are administered over a short period of time without the need for daily dosing. Moreover, if successful, immunologic memory is generated ensuring a destructive adaptive cellular immune response poised to eliminate aberrant cells at the time they first arise. T-memory cells, once vaccine primed, are active for years and can be boosted periodically with further vaccinations. In addition, vaccines have largely been shown to be non-toxic. There have been numerous clinical studies immunizing cancer bearing patients against proteins expressed in the colon with limited to no adverse events. Indeed, a single antigen vaccine targeting MUC-1 has progressed to clinical evaluation in patients with previous high-risk adenomas as a first attempt in CRC immuno-prevention. The vaccine was immunogenic, generating high levels of MUC-1 specific antibodies and few reported adverse effects. However, studies in transgenic mouse models have shown that multi-antigen vaccines are significantly more effective in inhibiting the progression of pre-invasive to invasive cancer and preventing clinical disease than any single antigen vaccine alone. In addition, prophylactic vaccines have had remarkable success in preventing cancers of viral origin such as hepatitis B and human papillomavirus, in part because the vaccines targeted proteins that drive oncogenesis. A major barrier to the extrapolation of the success seen with vaccine induced prevention of virally mediated malignancies to the prevention of CRC has been the lack of well-defined, biologically relevant, immunogenic proteins expressed early in the malignant transformation of the disease, e.g. cancer “initiation” antigens, that could be targeted in a multi-antigen vaccine.

There remains a need for a multi-antigen, multi-epitope vaccine targeting immunogenic proteins involved in CRC initiation that can be constructed and demonstrate both safety and efficacy.

### SUMMARY

The material described herein meets these needs and others by providing a vaccine in the form of a five-antigen poly-epitope DNA plasmid and related molecules designed to optimize antigenicity, immunogenicity and physicochemical properties of the encoded fusion protein. All proteins in the construct were confirmed to be expressed and vaccination generated high magnitude IFN-γ in the majority of animals evaluated. No toxicities attributable to vaccination were observed. Anti-tumor efficacy of vaccination is confirmed in treated mice. Thus, provided are, inter alia, a nucleic acid molecule comprising a nucleic acid sequence encoding a polypeptide comprising at least three epitopes selected from the group consisting of: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; (e) amino acids 880-895 and 962-976 of EGFR; and (f) an amino acid sequence having at least 90% identity with any one of the foregoing.

In some embodiments, the polypeptide comprises: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; and (e) amino acids 880-895 and 962-976 of EGFR. In some embodiments, the polypeptide comprises: (a) amino acids 124-164 of CDC25B; (b) amino acids 72-97 and 274-295 of COX2; (c) amino acids 30-119 of HIF-1alpha; (d) amino acids 93-164 of CDH3; and (e) amino acids 877-901 and 960-980 of EGFR. In some embodiments, the polypeptide comprises at least one of each of (a), (b), (c), (d), and (e), and optionally further comprises a linker sequence disposed between the epitopes. In some embodiments, the linker sequence is GGGGGS (SEQ ID NO: 16) or GPGPG (SEQ ID NO: 17). In some embodiments, the polypeptide has the amino acid sequence shown in SEQ ID NO: 18.

In some embodiments, the nucleic acid sequence comprises a promoter sequence. In some embodiments, the nucleic acid sequence comprises a CMV enhancer. In some embodiments, the nucleic acid sequence comprises a heterologous sequence. In some embodiments, the heterologous sequence encodes a promoter, a transcriptional start site, a translational start site, a detectable marker, a mRNA processing splice site, a polyadenylation sequence, and/or a regulatory element. In some embodiments, the nucleic acid sequence comprises the sequence shown in SEQ ID NO: 19.

Also provided is a vector comprising the nucleic acid molecule described above, wherein the vector is capable of directing expression of the encoded polypeptide(s). In some embodiments, the vector is a DNA plasmid, messenger RNA (mRNA), or viral vector. In some embodiments, the viral vector is a lentiviral vector, an adenoviral vector, or a poxviral vector. In some embodiments, the vector is mRNA. In some embodiments, the vector comprises the nucleotide sequence of SEQ ID NO: 19. In some embodiments, the vector encodes the polypeptide of SEQ ID NO: 18.

Further described herein is a polypeptide comprising at least three epitopes selected from the group consisting of: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; (e) amino acids 880-895 and 962-976 of EGFR; and (f) an amino acid sequence having at least 90% identity with any one of the foregoing. In some embodiments, the amino acid sequence has at least 95% identity with any one or more of the foregoing. In some embodiments, the polypeptide has the amino acid sequence shown in SEQ ID NO: 18.

Additionally described is a composition comprising the polypeptide, wherein the polypeptide comprises at least one of each of (a), (b), (c), (d), and (e), and optionally further comprises one or more linker sequences disposed between the epitopes. In some embodiments, the composition further comprises an adjuvant. In some embodiments, the polypeptide has the amino acid sequence shown in SEQ ID NO: 18.

Also described are methods for eliciting a Type 1 immune response directed at colorectal cancer, for preventing or treating colorectal tumors, and/or for ameliorating the symptoms of colorectal cancer in a subject in need thereof. In some embodiments, the method comprises administering to the subject a composition comprising the nucleic acid molecule or the composition described herein. In some embodiments, the Type 1 immune response is an IFN-gamma response.

In some embodiments, the subject is human. In some embodiments, the subject is suspected of having, or has been diagnosed with colorectal cancer.

### BRIEF DESCRIPTION OF THE DRAWINGS

FIGS. 1A-1D show immune responses for COLOVAC5. Plotted are IFN-γ corrected spots per well (cSPW) generated after immunization with (1A) COLOVAC5-1, (1B) COLOVAC5-2 or (1C) COLOVAC5-3 at the indicated dose and splenocytes stimulated with the single antigen peptide pool. (1D) Sum of all antigen IFN-γ cSPW after immunization with the indicated construct and dose level. All data are presented as box and whisker plots, showing all points, line at median and whiskers at minimum and maximum. * p<0.05, ** p<0.01, *** p<0.001, **** p<0.0001.

FIGS. 2A-2C are bar graphs showing the breadth of response in COLOVAC5. Percent of mice responding to the number of antigens after immunization with (2A) COLOVAC5-1, (2B) COLOVAC5-2 or (2C) COLOVAC5-3 at the indicated dose.

FIG. 3 provides a COLOVAC5.1 vector map generated at the GenScript website.

FIGS. 4A-4B show protein expression of antigens in COLOVAC constructs. (4A) Western blots of the indicated antigen after transfection of HEK-293 cells with the negative control sham construct (mock) or COLOVAC5-1, COLOVAC5-2 and COLOVAC5-3. (4B) Semi-quantitation of band density of each antigen relative to the loading control (tubulin) graphed as arbitrary units.

FIGS. 5A-5B show weight change in Study 1b was not significantly different between control and treated animals. (5A) Animal weights (mean±SEM) for Empty Vector (circles), Naproxen (squares), Colovac (triangles), and Colovac+Naproxen (inverted triangles) are shown from prior to start of vaccinations on day-2 until sacrifice at day 40. (5B) Overall change in weight from day 0 to day 40 for each animal is shown by treatment group.

FIGS. 6A-6B show that vaccination and naproxen synergize to reduce the number of intestinal lesions. The number of (6A) small intestine lesions in Apc^{Min/+} mice or (6B) colon lesions in AOM-treated mice after the indicated treatment presented as box and whisker plots showing all points, line at median and whiskers at minimum and maximum. INT: intermittent naproxen, CON: continuous naproxen *** p<0.001, **** p<0.0001.

FIGS. 7A-7D show dual naproxen and vaccine therapy increased CD8+ tumor infiltrating lymphocytes and reduced PD-L1 expression on tumors. Mean percent of CD8+ cells in total cells in tumors from (7A) Apc^{Min/+} mice or (7B) AOM-treated mice with the indicated vaccine and naproxen therapy presented as box and whisker plots showing all points, line at median and whiskers at minimum and maximum. Representative IHC from (7C) APCmin mice or (7D) mice treated with AOM. INT=intermittent, CON=continuous naproxen treatment. *p<0.05, ** p<0.01, *** p<0.001, **** p<0.0001.

FIGS. 8A-8H show the combination of vaccination with naproxen increased the antigen specific immune response which was associated with tumor protection. (8A) Corrected IFN-gamma spots per well (CSPW) and (8B) Granzyme B (pg/ml) levels from splenocytes stimulated with the vaccine peptide pool after the indicated in vivo treatment presented as box and whisker plots showing all points, line at median and whiskers at minimum and maximum in Apc^{Min/+} mice. Linear regression of tumor counts to (8C) IFN-gamma CSPW or (8D) Granzyme B in Apc^{Min/+} mice. (8E) IFN-gamma CSPW and (8F) Granzyme B (pg/ml) levels from splenocytes stimulated with the vaccine peptide pool after the indicated in vivo treatment presented as box and whisker plots showing all points, line at median and whiskers at minimum and maximum in AOM-treated mice. Linear regression of tumor counts to (8G) IFN-gamma CSPW or (8H) Granzyme B in AOM-treated mice. * p<0.05, ** p<0.01, *** p<0.001 **** p<0.0001.

FIGS. 9A-9H show tumor infiltrating lymphocyte biomarkers of the immune response with the combination of naproxen and vaccine can be identified. Receiver-operator curves when Apc^{Min/+} mice were divided by those receiving vaccine or naproxen alone and those receiving the combination therapy for (9A) CD8+CD3+, (AUC: 0.7623, p=0.002) (9B) Tbet+CD4+(AUC: 0.7273, p=0.008), (9C) EM CD4+ (AUC: 0.6782, p=0.041), (9D) EM CD8+ (AUC: 0.7897, p<0.001) tumor infiltrating lymphocytes. Receiver-operator curves when AOM-treated mice were divided by those receiving vaccine or naproxen alone and those receiving the combination therapy for (9E) CD8+CD3+ (AUC: 0.9296, p<0.0001), (9F) Tbet+CD4+ (AUC: 0.9051, p<0.0001), (9G) EM CD4+ (AUC: 0.8969, p<0.0001), (9H) EM CD8+ (AUC: 0.7469, p<0.001) tumor infiltrating lymphocytes.

### DETAILED DESCRIPTION

The invention described herein is based on the discovery and identification of several antigens that are involved in the progression of adenomas to colorectal carcinomas and are upregulated and overexpressed in these states, but not on normal colon tissue. Preliminary data demonstrated that immunization with COX2 and CDC25B can prevent the development of polyps in 50% of APC Min mice and significantly inhibit polyp formation in the remainder. The same vaccines resulted in a significant reduction in tumor formation in the AOM induced colon cancer model (p<0.05) with an EGFR vaccine also significantly inhibiting tumor formation (p<0.01). Additional antigens CDH3 and HIF1a are immunogenic and inhibit cancer formation in other tumor models. CDH3 and HIF1a are also overexpressed in adenomas and maintain overexpression to invasive colorectal carcinoma. Vaccine induced protection was mediated by CD8 T-cells. Combination immunoprevention focused on modulating both the polyp specific effector T-cell response (multi-antigen vaccine) while modulating the immune suppressive tumor microenvironment (NSAID therapy) provides a means for complete protection from the development of colon cancer.

### Definitions

All scientific and technical terms used in this application have meanings commonly used in the art unless otherwise specified. As used in this application, the following words or phrases have the meanings specified.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, a “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity to a target molecule or retains signaling activity. In certain embodiments, a linker is comprised of about two to about 35 amino acids, for instance, or about four to about 20 amino acids or about eight to about 15 amino acids or about 15 to about 25 amino acids.

As used herein, the terms “nucleic acid sequence” or “polynucleotide” refers to nucleotides of any length which are deoxynucleotides (i.e. DNAs), or derivatives thereof; ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA, DNA-RNA hybrids, oligonucleotides (oligos), or other natural, synthetic, modified, mutated or non-natural forms of DNA or RNA.

The term “vector” refers to, without limitation, a recombinant genetic construct or plasmid or expression construct or expression vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “multicistronic” or “polycistronic” or “bicistronic” or tricistronic″ refers to mRNA with multiple, i.e., double or triple coding areas or exons, and as such will have the capability to express from mRNA two or more, or three or more, or four or more, etc., proteins from a single construct. Multicistronic vectors simultaneously express two or more separate proteins from the same mRNA. The two strategies most widely used for constructing multicistronic configurations are through the use of 1) an IRES or 2) a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides.

As used herein, the terms “protein”, “peptide”, and “polypeptide” refer to amino acid subunits, amino acid analogs, or peptidomimetics. The subunits may be linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

The term “effective amount” or “therapeutically effective amount” or “prophylactically effective amount”, refer to an amount of an active agent described herein that is effective to provide the desired/intended result and/or biological activity. Thus, for example, in various embodiments, an effective amount of a composition described herein is an amount that is effective to result in remission or slowing the progression of disease, and/or to improve or to ameliorate symptoms of and/or to treat disease.

When the disclosure herein relates to a small molecule, polypeptide, protein, polynucleotide, nucleic acid, oligonucleotide, antisense, or miRNA, an equivalent or a biologically equivalent of such is intended within the scope of this disclosure. As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described herein. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1× SSC to about 0.1×SSC; formamide concentrations of about 55% to about 75%; and wash solutions of about 1× SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the compositions, therapy, nucleic acid molecules, and methods disclosed herein for inducing an immune response, beneficial or desired results can include one or more, but are not limited to, alleviation or amelioration of one or more symptoms of disease associated with colorectal cancer, delay or slowing of the condition (including disease), progression, amelioration or palliation of a condition (including disease), states of and remission of (whether partial or total) disease associated with colorectal cancer, whether detectable or undetectable.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, or otherwise in contact with compositions and/or nucleic acid molecules disclosed herein, include without limitation, cells of a human, non-human animal, mammal, or non-human mammal, including without limitation, cells of murine, canine, or non-human primate species. Cells treated, transfected, transformed, or otherwise in contact with compositions and/or nucleic acid molecules disclosed herein are, without limitation, T cells, antigen-presenting cells, and other suitable host cells.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “about,” as used herein when referring to a measurable value such as an amount, level or concentration, for example and without limitation, is meant to encompass variations of 20%, 10%, 5%, 1%, 0.5%, or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

The terms “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2, and VP3. Each particle contains approximately 5 VP1 proteins, 5 VP2 proteins and 50 VP3 proteins ordered into an icosahedral structure.

### Compositions and Nucleic Acid Molecules

Provided are compositions and/or nucleic acid molecules for preventing and treating colorectal cancer in a subject. Disclosed herein is a nucleic acid molecule comprising a nucleic acid sequence encoding a polypeptide comprising at least three epitopes selected from the group consisting of: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; (e) amino acids 880-895 and 962-976 of EGFR; and (f) an amino acid sequence having at least 90% identity with any one of the foregoing. In some embodiments, the nucleic acid sequence comprises the sequence shown in SEQ ID NO: 19.

In some embodiments, the polypeptide comprises: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; and (e) amino acids 880-895 and 962-976 of EGFR. In some embodiments, the polypeptide comprises: (a) amino acids 124-164 of CDC25B; (b) amino acids 72-97 and 274-295 of COX2; (c) amino acids 30-119 of HIF-1alpha; (d) amino acids 93-164 of CDH3; and (e) amino acids 877-901 and 960-980 of EGFR. In some embodiments, the polypeptide comprises at least one of each of (a), (b), (c), (d), and (e), and optionally further comprises a linker sequence disposed between the epitopes. In some embodiments, the linker sequence is GGGGGS (SEQ ID NO: 16) or GPGPG (SEQ ID NO: 17). In some embodiments, the polypeptide comprises the amino acid sequence shown in SEQ ID NO: 18.

Colovac 5.1 Protein Sequence
(SEQ ID NO: 18)
MAEQTFEQAIQAASRIIRNEQFAIRRFQSMPVRLLGHSPVLRGGGGGSNTVHYILTHEKG
FWNVVNNIPFLRNAGQEVFGLVPGLMMYATIWLREGGGGGSRSKESEVFYELAHOLPLPH
NVSSHLDKASVMRLTISYLRVRKLLDAGDLDIEDDMKAQMNCFYLKALDGFVMVLTDDGD
MIYISDNVNKYGGGGGSLKIFPSKRILRRHKRDWVVAPISVPENGKGPFPORLNQLKSNK
DRDTKIFYSITGPGADSPPEGVFAVEKETGGGGGSPIKWMALESILHRIYTHQSDVWSYG
KFRELIIEFSKMARDPQRYLV
Colovac 5.1 DNA sequence
(SEQ ID NO: 19)
ATGGCCGAGCAGACCTTCGAGCAGGCCATCCAGGCCGCCAGCAGGATCATCCGCAACGAGCAGTTCGCCATCCGG
AGATTTCAGAGCATGCCAGTGAGGCTGCTGGGACACTCCCCCGTGCTGAGGGGAGGAGGAGGAGGCTCCAATACC
GTGCACTACATCCTGACACACTTCAAGGGCTTTTGGAACGTGGTGAACAATATCCCTTTCCTGCGGAATGCCGGC
CAGGAGGTGTTTGGCCTGGTGCCAGGCCTGATGATGTACGCCACAATCTGGCTGAGGGAGGGAGGAGGAGGAGGA
TCTAGAAGCAAGGAGTCTGAGGTGTTTTATGAGCTGGCACACCAGCTGCCACTGCCACACAACGTGAGCTCCCAC
CTGGACAAGGCCTCTGTGATGCGGCTGACCATCAGCTACCTGCGGGTGAGAAAGCTGCTGGATGCCGGCGACCTG
GACATCGAGGACGATATGAAGGCCCAGATGAATTGCTTCTATCTGAAGGCCCTGGACGGCTTTGTGATGGTGCTG
ACAGACGATGGCGACATGATCTACATCTCCGATAACGTGAATAAGTATGGCGGCGGCGGCGGCAGCCTGAAGATT
TTCCCATCCAAGAGGATCCTGAGGCGCCACAAGAGGGACTGGGTGGTGGCACCCATCTCCGTGCCTGAGAACGGC
AAGGGCCCTTTTCCACAGCGGCTGAACCAGCTGAAGTCTAATAAGGACAGAGATACCAAGATTTTCTACTCCATC
ACAGGACCAGGAGCAGATTCTCCACCTGAGGGCGTGTTTGCAGTGGAGAAGGAGACTGGAGGAGGAGGAGGCTCT
CCTATCAAGTGGATGGCCCTGGAGAGCATCCTGCACAGGATCTACACACACCAGTCCGACGTGTGGTCTTATGGC
AAGTTCCGCGAGCTGATCATCGAGTTTAGCAAGATGGCCCGGGATCCTCAGAGATACCTGGTG

In some embodiments, the nucleic acid sequence comprises a promoter sequence. In some embodiments, the nucleic acid sequence comprises a CMV enhancer. In some embodiments, the nucleic acid sequence comprises a heterologous sequence. In some embodiments, the heterologous sequence encodes a promoter, a transcriptional start site, a translational start site, a detectable marker, a mRNA processing splice site, a polyadenylation sequence, and/or a regulatory element.

TABLE 1
Peptide sequences

| | | | Human/mouse |
|---|---|---|---|
| Antigen | Length | Sequence (SEQ ID NOs: 1-10) | homology (peptide) |
| CDC25B | P132-156 | IQAASRIIRNEQFAIRRFQSMPVRL | 76% |
| COX2 | P76-93 | YILTHFKGFWNVVNNIPF | 89% |
| | P276-295 | EVFGLVPGLMMYATIWLREH | 100% |
| EGFR | P880-895 | WMALESILHRIYTHQS | 100% |
| | P962-976 | RELIIEFSKMARDPQ | 93% |
| CDH3 | P93-110 | LKIFPSKRILRRHKRDWV | 56% |
| | P137-156 | RDTKIFYSITGPGADSPPEG | 95% |
| HIF1A | P38-53 | YELAHQLPLPHNVSSH | 100% |
| | P60-82 | MRLTISYLRVRKLLDAGDLDIED | 91% |
| | P93-117 | LKALDGFVMVLTDDGDMIYISDNVN | 96% |

In some embodiments, the nucleic acid sequence encodes a polypeptide that comprises a combination of one or more epitopes. In some embodiments, the encoded polypeptide comprises one or more of the epitopes listed

In some embodiments, the nucleic acid sequence optionally further comprises one or more linker sequences disposed between the epitopes. In some embodiments, the linker sequence is glycine-serine. In some embodiments, the nucleic acid sequence comprises a promoter sequence. In some embodiments, the nucleic acid sequence comprises a heterologous sequence. In some embodiments, the heterologous sequence encodes a promoter, a transcriptional start site, a translational start site, a detectable marker, a mRNA processing splice site, a polyadenylation sequence, and/or a regulatory element.

Such nucleic acid molecules may be delivered by viral or non-viral means. One example of viral delivery is adeno-associated virus (AAV). Other examples include retrovirus, lentivirus, and baculovirus delivery. One example of a non-viral method of delivery is cell penetrating peptide (CPP). Polynucleotide constructs may also be modified, such as through chemical modification, to improve their stability and/or suitability for delivery. In some embodiments, the oligonucleotide is modified by locked nucleic acids and/or phosphorothioate linkages. In some embodiments, a delivery system is selected for improved bioavailability, such as PEGylated liposomes, lipidoids, or biodegradable polymers, as examples.

### Viral Vectors

In some embodiments, the vector disclosed herein is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers.

In some embodiments, the vector disclosed herein is an AAV vector with low toxicity. In some embodiments, the AAV vector does not incorporate into the host genome, thereby having a low probability of causing insertional mutagenesis. In some embodiments, the AAV vector can encode a range total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof.

In some embodiments, the vector disclosed herein is a lentiviral vector. In one embodiment, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art. In some embodiments, exemplary lentiviral vectors that may be used in relation to any of the herein described compositions, nucleic acid molecules and/or methods, and can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, a equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and/or nucleic acid molecules and/or methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and/or nucleic acid molecules and/or methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

In some embodiments, expression vector or viral vector disclosed herein is used to transfect, transform, or come in contact with a cell which is a eukaryotic cell. In some embodiments, the cell is an animal cell. In some embodiments, the cell is a mammalian cell. In some embodiments, the cell is a bovine, murine, feline, equine, porcine, canine, simian, or human cell.

In some embodiments, a cell is a packaging cell or a producer cell for production of a viral particle.

In some embodiments, provided herein are viral particles comprising, consisting of, or consisting essentially of a vector comprising, consisting of, or consisting essentially of a polynucleotide sequence encoding one or more polypeptides as described herein.

In general, methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid-based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the above env genes or chimeric envelope genes encoding the cytoplasmic and transmembrane of the above env gene products and a monoclonal antibody directed against a specific surface molecule on a desired target cell. Similar vector-based systems may employ other vectors such as sleeping beauty vectors or transposon elements.

The resulting packaged expression systems may then be introduced via an appropriate route of administration, discussed in detail with respect to the method aspects disclosed herein.

### Polypeptides

Further described herein is a polypeptide comprising at least three epitopes selected from the group consisting of: (a) amino acids 132-156 of CDC25B; (b) amino acids 76-93 and 276-295 of COX2; (c) amino acids 38-53, 60-82, and 93-117 of HIF-1alpha; (d) amino acids 93-110 and 137-156 of CDH3; (e) amino acids 880-895 and 962-976 of EGFR; and (f) an amino acid sequence having at least 90% identity with any one of the foregoing. In some embodiments, the epitopes selected include an amino acid sequence having at least 90% identity with any one of the amino acid sequences listed in Table 1. In some embodiments, the polypeptide comprises a combination of at least three, four, five, six, or more of the epitopes listed above in Table 1. In some embodiments, the polypeptide comprises at least one of each of (a), (b), (c), (d), (e), and (f). In some embodiments, the polypeptide further comprises one or more linker sequences disposed between the epitopes.

### Pharmaceutical Compositions

Pharmaceutical compositions disclosed herein include one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for administration as a vaccine, for example, with the inclusion of an adjuvant.

For example, in some embodiments, provided is a composition comprising the polypeptide, wherein the composition further comprises an adjuvant. In some embodiments, the adjuvant elicits T cell responses. In some embodiments, the composition comprises a nucleic acid molecule disclosed herein.

### Methods of the Invention

Described herein are methods for eliciting a Type 1 immune response directed at colorectal cancer, for preventing or treating colorectal tumors, and/or for ameliorating the symptoms of colorectal cancer in a subject in need thereof. In some embodiments, the method comprises administering to the subject a composition comprising the nucleic acid molecule or the composition described herein. In some embodiments, the Type 1 immune response is an IFN-gamma response. In a typical embodiment, the method comprises administering to a subject in need thereof a composition as described herein.

### Administration and Dosage

The compositions and/or nucleic acid molecules disclosed herein are administered in any suitable manner, often with pharmaceutically acceptable carriers. Suitable methods of administering compositions, compounds, molecules, nucleic acids, and vectors in the context of the present invention to a subject are available, and, although more than one route can be used to administer a particular composition, a particular route can often provide a more immediate and more effective reaction than another route.

The dose administered to a patient, in the context of the disclosure herein, should be sufficient to result in a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

Routes, order and/or frequency of administration of the therapeutic compositions disclosed herein, as well as dosage, will vary from individual to individual, and may be readily established using standard techniques. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1: a Multi-Antigen DNA Vaccine Encoding Antigen Epitopes Included in the Peptide-Based Colovac Vaccine

A vaccine approach is well suited to CRC prevention as a limited number of immunizations can be given over a short period of time to achieve a desired immune response. Further, if T-cells are appropriately activated by a vaccine the cells can traffic to antigen aberrantly expressed in neoplastic tissue anywhere in the body. Most importantly if an immunologic memory response develops, antigen specific T-cells will persist in the body for years ready to be deployed when aberrant cells are detected.

We identified several antigens that are involved in the progression of adenomas to colorectal carcinomas and are upregulated and overexpressed in these states, but not on normal colon tissue. Preliminary data demonstrated that immunization with COX2 and CDC25B can prevent the development of polyps in 50% of APC Min mice and significantly inhibit polyp formation in the remainder. The same vaccines resulted in a significant reduction in tumor formation in the AOM induced colon cancer model (p<0.05) with an EGFR vaccine also significantly inhibiting tumor formation (p<0.01). Additional antigens CDH3 and HIF1a are immunogenic and inhibit cancer formation in other tumor models. CDH3 and HIF1a are also overexpressed in adenomas and maintain overexpression to invasive colorectal carcinoma. Vaccine induced protection was mediated by CD8 T-cells. Combination immunoprevention focused on modulating the polyp specific effector T-cell response (multi-antigen vaccine) while also modulating the immune suppressive tumor microenvironment (NSAID therapy), which may result in complete protection from the development of colon cancer.

We set out to evaluate the immunogenicity of all permutations of Colovac-5 (n=320) by in situ analyses with linkers (-AAY-, -GGGGGS- and -GPGPG-) and alternate antigen order. Sequences were evaluated for antigenicity (ddg-pharmfac.net/vaxijen/VaxiJen/VaxiJen.html). The constructs with the -AAY-linker, regardless of antigen order, did not meet the minimum antigenicity score of 0.5, so the constructs with this linker were not considered further. We have previously observed that constructs with the highest CD4 immunogenicity scores (tools.iedb.org/CD4episcore) are more immunogenic than constructs with lower scores. Thus, the remaining constructs were ranked from the highest to lowest immunogenicity score. To further refine the candidate constructs, we examined various predicted physiochemical properties including instability index, solubility, half-life (web.expasy.org/protparam) and secondary structure (bioinf.cs.ucl.ac.uk/psipred) from the top 15 constructs with the goal of choosing three final candidates. Instability index and solubility were similar for all constructs so those parameters were not considered further. The three constructs chosen for Colovac-5 are listed Table 2. Construct 5-1 is the first ranked construct based on immunogenicity score and is predicted to have a short protein half-life and 38% of the epitopes in a helical secondary structure. Half-life and secondary structure was not predicted to be different until the sixth-ranked construct (Colovac-5.2). This protein also was predicted to have a short half-life and only 26% of the protein in a helical secondary structure. The next predicted half-life and secondary structure that was different is in the fourteenth-ranked construct (Colovac-5.3). This protein was predicted to have longer half-life and more of the protein predicted to be in a helical secondary structure (44%) as compared to the other two the candidate constructs. We hypothesized that a longer half-life of a candidate protein is positively correlated to increased immunogenicity. Additionally, it has been reported that epitopes that are predicted to generate proteins in helical secondary structure are more immunogenic than epitopes predicted to be in protein sheets. Thus, with further analysis of these three candidates, we aim to determine which in situ prediction parameter(s) correlates most with immunogenicity.

TABLE 2
Multi-Antigen DNA Vaccines

| | | Antigen | | | | | | | |
|---|---|---|---|---|---|---|---|---|---|
| | | Sequence | | | | | | | |
| | GenScript | (“-” = | | | In | In | | | |
| | DNA | GGGGGS | Anti- | Immuno- | Vitro | vivo | | | |
| Lab | synthesis | linker; SEQ | genicity | genicity | half | half | | Instability | % in |
| Name | name | ID NO: 16) | Index | Score | life | life | | index | helix |
| CV5.1 | COLOVAC5- | CDC25B- | 0.6278 | 1762 | 4.4 h | >20 | h | 62.05 | 45% |
| | 005 | COX2- | | | | | | | |
| | | HIF1a- | | | | | | | |
| | | CDH3- | | | | | | | |
| | | EGFR | | | | | | | |
| CV5.2 | COLOVAC5- | CDH3- | 0.6544 | 1819 | 5.5 h | 3 | min | 62.05 | 25% |
| | 096 | CDC25B- | | | | | | | |
| | | HIF1a- | | | | | | | |
| | | COX2- | | | | | | | |
| | | EGFR | | | | | | | |
| CV5.3 | COLOVAC5- | HIF1a- | 0.623 | 1919 | 1 h | 2 | min | 61.45 | 35% |
| | 117 | COX2- | | | | | | | |
| | | CDH3- | | | | | | | |
| | | CDC25B- | | | | | | | |
| | | EGFR | | | | | | | |

TABLE 3
Epitopes Included in DNA Vaccines comprising COLOVAC constructs

| | | | Human- |
|---|---|---|---|
| | Amino Acid | | mouse |
| Antigen | locations | Sequence (SEQ ID NOs: 11-15) | homology |
| CDC25B | 124-164 | AEQTFEQAIQAASRIIRNEQFAIRRFQSMPVRLLGHSPVLR | 78% |
| COX2 | 72-97, 274- | NTVHYILTHFKGFWNVVNNIPFLRNAGQEVFGLVPGLMMYATI | 91% |
| | 295 | WLRE | |
| EGFR | 877-901, | PIKWMALESILHRIYTHQSDVWSYGKFRELIIEFSKMARDPQR | 98% |
| | 960-980 | YLV | |
| CDH3 | 93-164 | LKIFPSKRILRRHKRDWVVAPISVPENGKGPFPQRLNQLKSNK | 86% |
| | | DRDTKIFYSITGPGADSPPEGVFAVEKET | |
| HIF1A | 30-119 | RSKESEVFYELAHQLPLPHNVSSHLDKASVMRLTISYLRVRKL | 94% |
| | | LDAGDLDIEDDMKAQMNCFYLKALDGFVMVLTDDGDMIYISD | |
| | | NVNKY | |

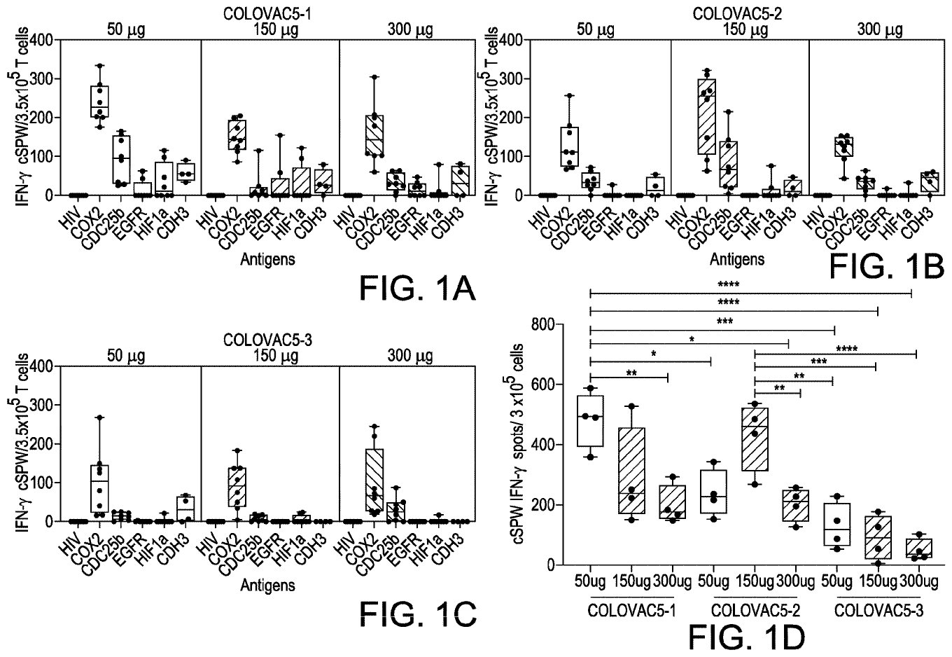

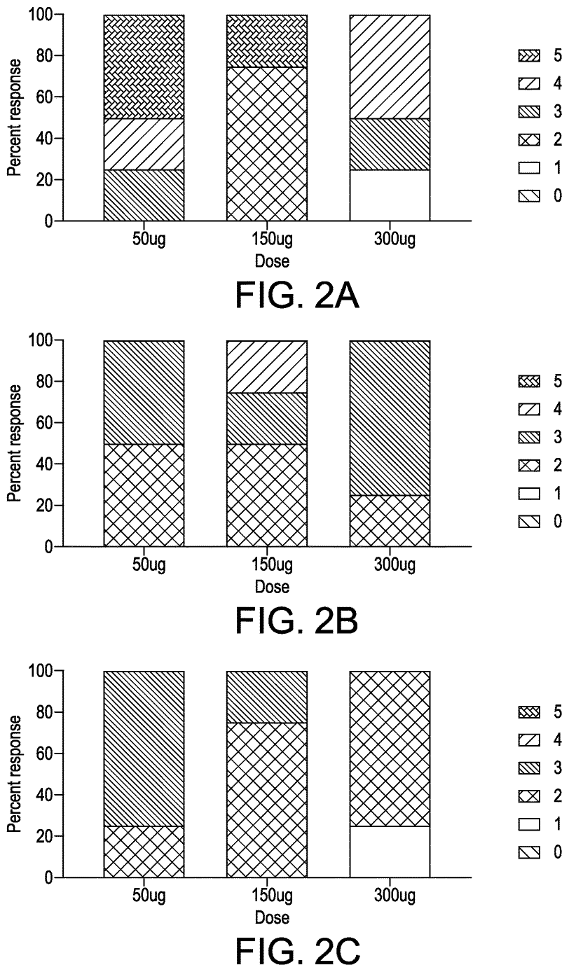

Potential differences in immune response obtained with different antigen orders outlined in Table 2 were investigated. FIG. 1 shows the IFN-γ ELISPOT data for each of the three COLOVAC5 constructs. All antigens are immunogenic and there was 100% incidence to any construct, with a “responder” defined as a mouse that generated a significant IFN-γ spot count to at least one antigen. We used the sum of spot count to determine which construct was the most robust (FIG. 1D). COLOVAC5-1 and COLOVAC5-2 generated a higher magnitude IFN-γ response than COLOVAC5-3 (p<0.05 for all, FIG. 1C). COLOVAC5-1 was the most potent construct of the two. There was a significantly greater response at the lowest dose (50 ug) of COLOVAC5-1. COLOVAC5-2 did generate an equivalent magnitude immune response as observed in COLOVAC5-1, but at three times the dose. Furthermore, the breadth of response for COLOVAC5-1 was greater than COLOVAC5-2 (FIG. 2A, 2B).

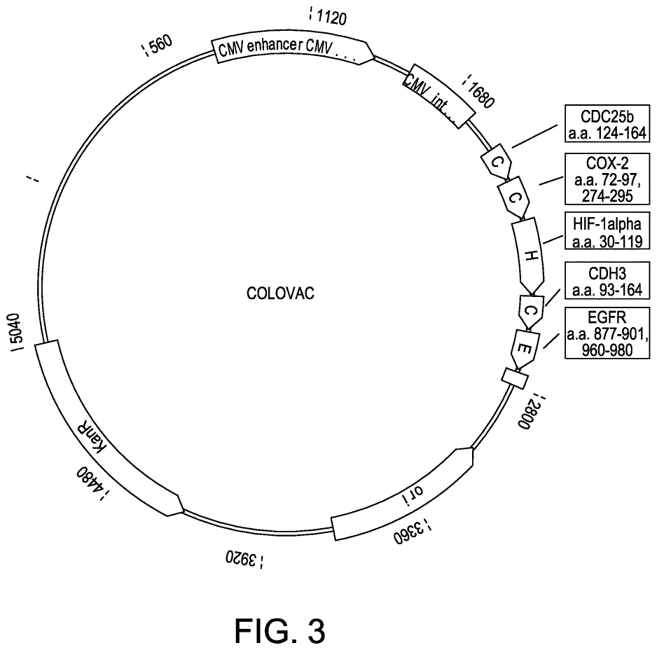

Based on these data, we decided to use COLOVAC5-1 at the 50 ug dose as the final candidate. The vector map for our optimal vaccine, COLOVAC-5.1, is shown in FIG. 3.

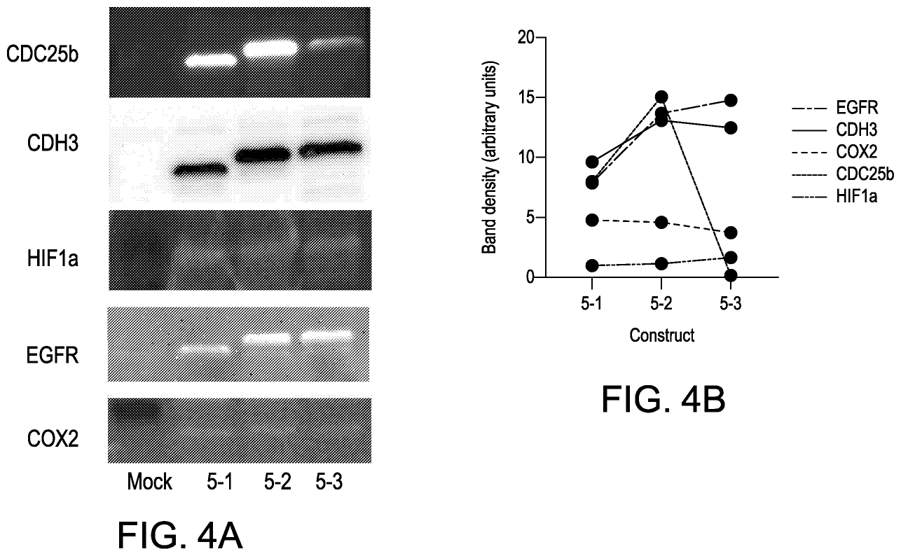

Expression of all five antigens of COLOVAC-5.1, 5.2, and 5.3 (CDC25B, CDH3, HIF1a, EGFR and COX2) has been confirmed in HEK-293 cells by Western Blot (FIG. 4).

### Example 2: Safety of the Multi-Antigen Colovac DNA Vaccine

A toxicity study was performed, as was an evaluation of immunogenic responses to each antigen. Animals were vaccinated (groups described below), every 10-14 days for four total vaccines, using 5 ug GM-CSF as an adjuvant. Multi-antigen acute toxicity treatment groups, n=10 FVB mice/group were treated with either 50 ug Empty Vector+GM-CSF, ID; or 50 ug COLOVAC5.1+GM-CSF, ID.

Multi-antigen acute immunogenicity treatment groups, FVB mice, were treated with: 150 ug Empty Vector+GM-CSF, ID (n=5); 50 ug COLOVAC5.1+GM-CSF, ID (n=10); 100 ug COLOVAC-3 peptide pool+GM-CSF, SC (n=10); or PBS+GM-CSF, SC (n=5). Animals were sacrificed roughly 10 days after the 4th vaccination. We then collected ¾ of each spleen and processed to cell suspension. IFNγ ELISPOT was performed to evaluate immunogenic responses to each antigen (pooled peptides). We predicted that the average response to vaccine would be at least 90 spots per well with a common standard deviation of 60. At these response levels, a sample size of 10 mice per group achieves 90% power to detect a significant difference between antigen-stimulated wells and HIV or no antigen-stimulated wells.

This experiment showed significant IFNγ spots in both COLOVAC 5.1 (p=0.019) and the three-antigen peptide mix (p=0.009) as a sum response to the five COLOVAC antigens in FVB mice.

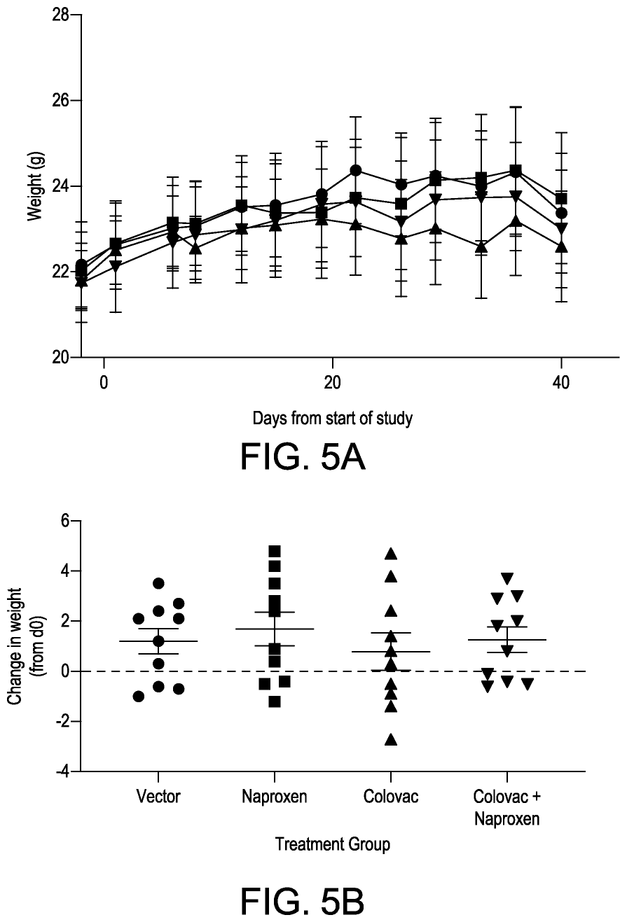

No significant differences were observed in the weight change or overall weight gain in any one treatment group when compared to empty vector control (FIG. 5).

Complete Blood Count parameters showed no significant differences between selected groups compared, as determined by t-test at p=0.01 (p-value adjusted for multiple comparisons), except for percent Eosinophils (p=0.00774). We determined that COLOVAC-5.1 treated animals had significantly lower percent Eosinophils than Vector treated animals. Serum Chemistry parameters showed no significant differences between selected groups compared, as determined by t-test at p=0.01 (p-value adjusted for multiple comparisons).

No significant findings or abnormalities were observed in any group during gross assessment (n=6/group). A t-test was performed across select comparison groups and interpretation was corrected for multiple comparisons. There were no significant differences in major organ weights for group compared, for any organ. Carcasses and ¼ spleen were collected and stored in 10% formalin.

This Example shows that Colovac-5.1 was immunogenic in 100% of the mice evaluated. The mean precursor frequency of Colovac5.1-specific T-cells after vaccination was 1:724. Seventy-five percent of the mice developed an IFN-γ immune response to at least four antigens with 50% of mice developing a response to all five antigens. Colovac-5.1 was safe in all parameters assessed, evaluated at both an acute and chronic time point.

### Example 3: Vaccination with Colovac Reduces the Number of Intestinal Lesions

This example demonstrates the reduction of intestinal tumors in a mouse model of colorectal cancer with administration of Colovac, both with and without naproxen coadministration. In addition, this therapy increased CD8+ tumor infiltrating lymphocytes and reduced PD-L1 expression on tumors, and increased the antigen specific immune response which was associated with tumor protection.

### MATERIALS AND METHODS

Animal models. Work was performed in accordance with the University of Washington Animal Care and Use Committee guidelines in a Specific Pathogen Free, PHS assured (#A3464-01), AAALAC accredited (#000523) environment. APC Min [Strain name: C57BL/6J-ApcMin/J, stock #002020] males, C57BL6/J females [stock #000664], and FVB/nJ [stock #001800] mice were purchased from Jackson Laboratory. The offspring from mated APC Min males and C57BL6/J females were genotyped by PCR for the presence of the Min mutation using primers as follows: Wild-Type: 5′-GCCATCCCTTCACGTTAG-3′, Common: 5′-TTCCACTTTGGCATAAGGC-3′, Mutant: 5′-TCCTGAGAAAGACAGAAGTTA-3′. For the AOM model, FVB/nJ mice were acclimated for one week before initial vaccinations. AOM (Sigma-Aldrich) was administered 10 mg/kg twice a week via intraperitoneal (ip) injection for six weeks. [1] Animals were housed 2 to 5 mice per cage, tested for specific pathogens via air dust routinely (quarterly or more), and housed on BED-O-COB bedding as previously reported (PMID: 36123012).

Vaccine. The vaccine consisted of the Th1-selective epitopes CDC25B-p132-156, COX2-p76-93, COX2-p276-295, EGFR-p880-895, and p962-976. The Th selective epitopes were determined in human PBMC screening by calculating the ratio of the product of the mean magnitude and percent incidence of response for IFN-γ and IL-10 ELISPOT assays (IFN-γ incidence X magnitude/IL-10 incidence X magnitude) [2]. Ratios greater than 7 defined Th1-selective epitopes (See Table 4).

TABLE 4
Peptide sequences of CDC25B, COX2, and EGFR

| | | Mouse | |
|---|---|---|---|
| Peptide | Sequence (SEQ ID NOs: 1-5) | homology | Th ratio |
| CDC25b-p132-156 | IQAASRIIRNEQFAIRRFQSMPVRL | 72% | 186 |
| COX-2-p76-93 | YILTHFKGFWNVVNNIPF | 89% | 7 |
| COX2-p276-295 | EVFGLVPGLMMYATIWLREH | 100% | 17 |
| EGFR-p880-895 | WMALESILHRIYTHQS | 100% | 11 |
| EGFR-p962-976 | RELIIEFSKMARDPQ | 100% | 41 |

Vaccination, Naproxen administration and assessment of tumor growth. Mice were immunized subcutaneously with 50 μg each peptide in a pool (CPC Scientific) with equal volume of adjuvant Control mice received adjuvant alone. An untreated control group was included for comparison to adjuvant alone; all results reported were similar between untreated control and adjuvant alone control groups. Complete Freund's Adjuvant was used in the first immunization, followed by Incomplete Freund's Adjuvant (Sigma-Aldrich) for subsequent immunizations.

Naproxen was mixed with meal-form, irradiated chow (PicoLab Rodent Diet 20) at a final concentration of 400 parts per million (ppm), then mixed with autoclaved drinking water and formed into pellets. After drying overnight in empty cages with filtered air via Allentown racks, pellets were provided to mice ad libitum. Samples were confirmed to have ≥94% of the intended concentration of compound by high pressure liquid chromatography.

The entire colon from the AOM model or entire small intestine from ApcMin/+ mice were removed at sacrifice and cut longitudinally with dissection scissors. Fecal matter was washed off and polyps were counted macroscopically by the same operator for each experiment. Data are expressed as the total number of lesions in each intestinal tissue.

In vivo study design. Both male and female mice were included in the study. For the ApcMin/+ study, animals were randomized into treatment groups sequentially after PCR-confirmed genotype at 5 weeks of age. Vaccination was administered at the start of study and every 7-10 days for a total of 4 doses. Naproxen was provided concurrently with vaccination, with some mice receiving naproxen continuously and some intermittently (3 weeks on and 3 weeks off). Mice in the vaccine treatment groups received a booster vaccine at 9 weeks old and the study was terminated when the mice were 22±2 weeks old. For the AOM study, animals were randomized into treatment groups sequentially at 6 weeks of age. Vaccination was administered at the start of study and every 10-14 days for a total of 3 doses. AOM was initiated 3-5 days after the third dose of vaccine. Booster doses of vaccine were administered upon the completion of AOM treatment (age 15 weeks) and again at 21 weeks. Naproxen was provided as described with the ApcMin/+ study. The study was terminated when the mice were 27±1 weeks old. The investigators and animal caretakers were not blinded to the treatment groups.

For ApcMin/+ animals, 14 mice per group were predicted to achieve ≥93% power to detect a difference in small bowel (SB) tumors of 33 tumors, assuming a common standard deviation of 25 tumors. For AOM treated mice, 14 mice per group were predicted to achieve ≥94% power to detect a difference of 4 colon tumors, assuming a common standard deviation of 3 tumors.

All data points for these experiments are included in the analysis with the following exceptions: In the ApcMin/+ experiment, 14 mice per group, seven males and seven females, were included originally. Of these, one mouse in the vaccine alone group was found dead one week prior to scheduled endpoint, and excluded from all analyses. In the AOM experiment, 14 mice per group, seven males and seven females, were included originally. Of these, two mice in the adjuvant control group, three mice in the adjuvant+ CON group, six mice in the vaccine alone group, and four mice in each vaccine+ INT and vaccine+ CON groups were excluded from analysis due to early termination or death. The number of mice analyzed is indicated in the figure legend.

Analysis of peptide-specific T-cell responses. Mouse splenic cells were evaluated by ELISPOT for antigen-specific IFN-gamma (γ) as previously described [3]. The ELISPOT used 10 μg/ml each of experimental peptide, the negative control peptide HIVp52 and the positive controls of Concanavalin A (Sigma). Data for are reported as corrected spots/well (cSPW) which is the mean number of spots for each experimental antigen minus the mean number of spots detected in no antigen control wells. Granzyme B was evaluated via ELISA (Invitrogen) according to the manufacturer's instructions from the supernatant from each ELISPOT. Data are reported as pg/ml.

Immunophenotyping. After removing up to 10 polyps in ApcMin/+, and 1-2 polyps in AOM treated mice, remaining tumors were removed from the intestinal tissue with forceps and applied to a 70 μm filter, washed through with flow cytometry buffer (PBS/1% FBS/2 mM EDTA) and red cells lysed. 0.5×106 viable cells from samples were blocked with anti-mouse CD16/32 (Mouse BD Fc Block, clone 2.4G2). Prior to surface staining, cells were stained with Fixable viability dye 450 (eBiosciences; 1:1000) in PBS and then washed in flow cytometry buffer. Intracellular staining was performed using the FOXP3 buffer set (Thermo Fisher Scientific). Antibodies were obtained from BD Bioscience, Biolegend or Thermo Fisher Scientific. Receptor expression was documented by incubating with anti-mouse-CD3e (PE or APC; clone 145-2C11), -Tbet (PE/Cy7; clone 4B10), -GATA3 (PE, clone; TWAJ), -CD4 (PE/Cy5; clone GK1.5), -CD62L (APC; clone MEL-14), -FOXP3 (AlexaFluor488; clone FJK-16s), -CD44 (FITC; clone IM7), -CD8a (PE/Cy7; clone 53-6.7), -PD-1 (APC-Cy7, clone J43), or the appropriate isotype control for 1 hour at 2-8° C. protected from light. Analysis was performed on FACS Canto RUO and data analyzed using FlowJo software (BD Biosciences). Typically, 2×105 events were recorded from the viability gate per sample. Results are reported as a percentage of total cell number or a percentage of a specific cell population.

Immunohistochemistry. Tumors removed from the intestine were covered with Tissue-Tek O.C.T Compound in a cryomold and stored at −80° C. Frozen blocks were sectioned and fixed with 75% acetone/25% methanol for 5 minutes. The slides were washed thrice with PBS and 10% goat serum was added for 1 hour at room temperature followed by an anti-mouse PD-L1 (abcam; clone: MIH6; 1/100 dilution) or anti-mouse CD8 (AbD Serotec; clone KT15; 1/200 dilution) overnight at 4° C. After washing, the slides were incubated with AlexaFluor 488 anti-rat IgG (abcam; 1:1000 dilution) for 1 hour at room temperature. Positive cells were counted in three 20× microscopic fields and expressed as the mean percent of total cells.

Statistical analysis. Model assumptions were checked using the Shapiro-Wilk normality test and by visual inspection of residual and fitted value plots. The unpaired, two-tailed Student's t-test and ANOVA test was used to evaluate differences when normality was confirmed. When normality of the data was not confirmed, the non-parametric Kruskal-Wallis and Man-Whitney tests were used. Differences in tumor volume was determined by two-way ANOVA with a Dunnett post-test for multiple comparisons. A p value of <0.05 was considered significant (GraphPad Software, Prism v.8).

### RESULTS

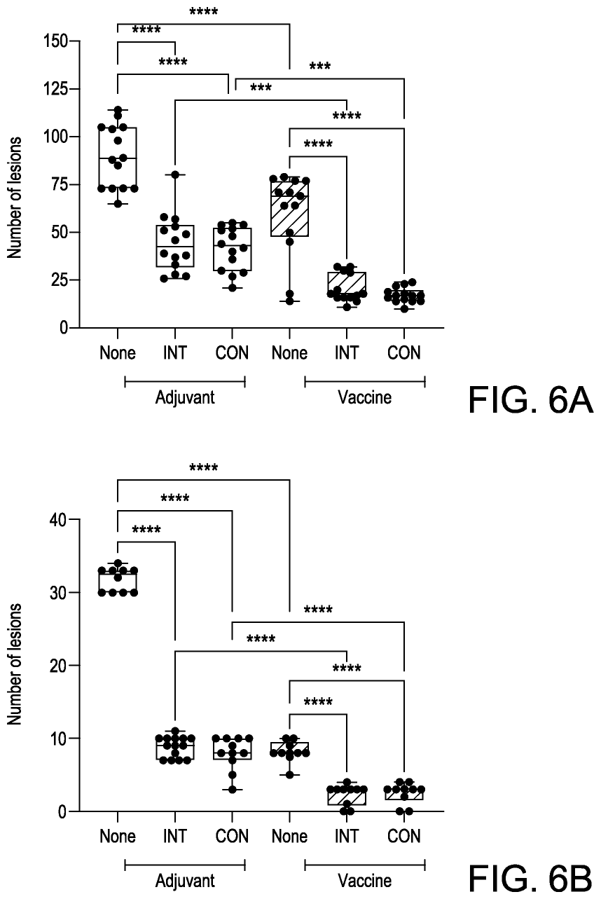

Vaccination and naproxen synergize to reduce the number of intestinal lesions. As expected in the ApcMin/+ mice [4], fewer small bowel tumors were identified in mice treated with continuous naproxen (mean, 41.6±2.5 lesions) as compared to untreated mice (mean, 89.7±3.7 lesions; p<0.001; FIG. 6A). Mice treated with intermittent naproxen also exhibited fewer lesions than untreated mice (mean, 44.4±3.3 lesions; p<0.0001), which was no different than mice treated with continuous naproxen (p=0.99; FIG. 6A). We observed significantly fewer tumors in mice immunized with the vaccine (mean, 59.7±3.3 lesions) as compared to the sham vaccinated mice (mean, 89.7±3.7 lesions; p<0.0001; FIG. 6A). Significantly fewer tumors were observed when immunized mice were also treated with either intermittent (mean, 20.7±2 lesions) or continuous (mean, 17.1±1 lesions) naproxen as compared to either monotherapy alone (p<0.001 for all; FIG. 6A). A similar result was observed in AOM-treated mice. Fewer small bowel tumors were identified in mice treated with continuous (mean, 6±0.6 lesions) or intermittent (mean, 8.9±0.4 lesions) naproxen as compared to untreated mice (mean, 31.6±0.5 lesions; p<0.0001; FIG. 6B). We also observed significantly fewer tumors in mice immunized with the vaccine (mean, 8.2±0.5 lesions) as compared to the sham vaccinated mice (p<0.0001; FIG. 6B). Significantly fewer tumors were observed when immunized mice were also treated with either intermittent (mean, 2.3±0.5 lesions) or continuous (mean, 2.5±0.5 lesions) naproxen as compared to either monotherapy alone (p<0.0001 for all; FIG. 6B).

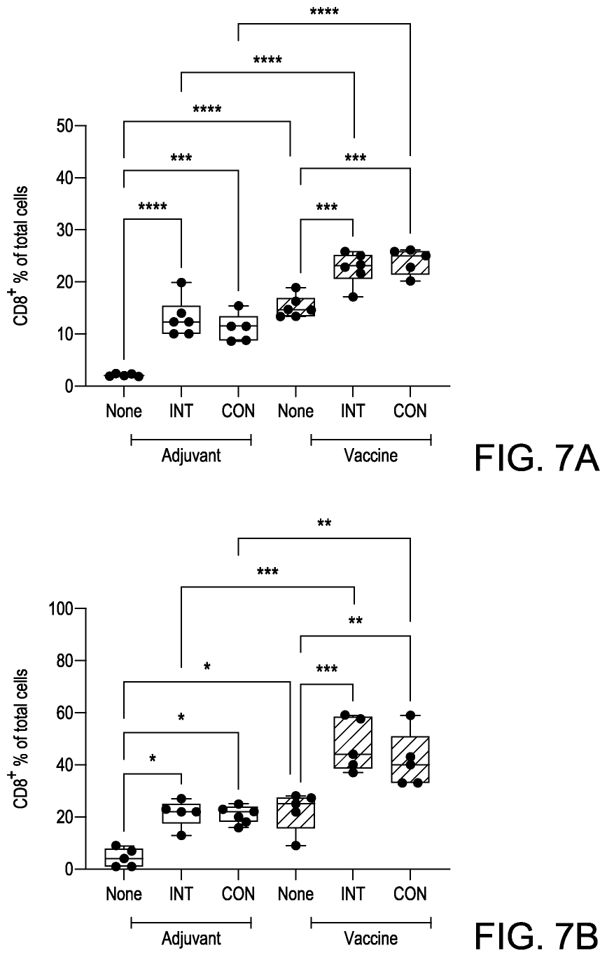

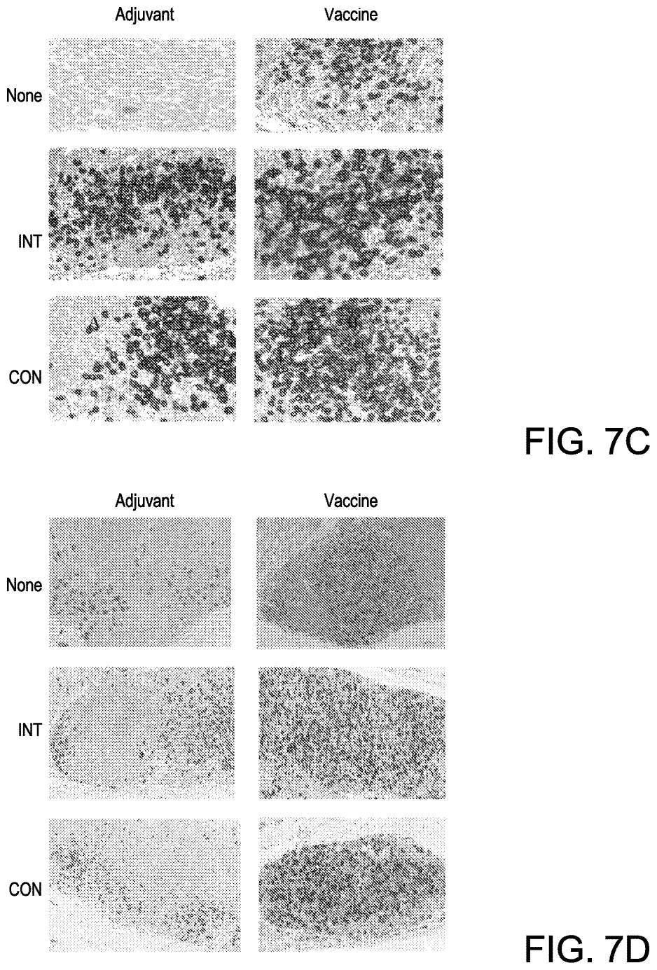

Dual naproxen and vaccine therapy increased CD8+ tumor infiltrating lymphocytes and reduced PD-L1 expression on tumors. We have previously observed that tumor cell expression of PD-L1 was reduced and tumor infiltrating CD8+ T-cells was increased when ApcMin/+ mice were treated with naproxen [4], which we confirm in this study with naproxen administered either intermittently or continuously (p<0.001 for both; FIG. 7A, B). Vaccine alone increased tumor infiltrating CD8+ T-cells by 86% as compared to control (p<0.0001; FIG. 7A), but treating with naproxen at either regimen further increased tumor infiltrating CD8+ T-cells by at least 32% as compared to either therapy alone (p<0.001 for all; FIG. 7A). A similar result was observed for mice treated with AOM (FIG. 7B).

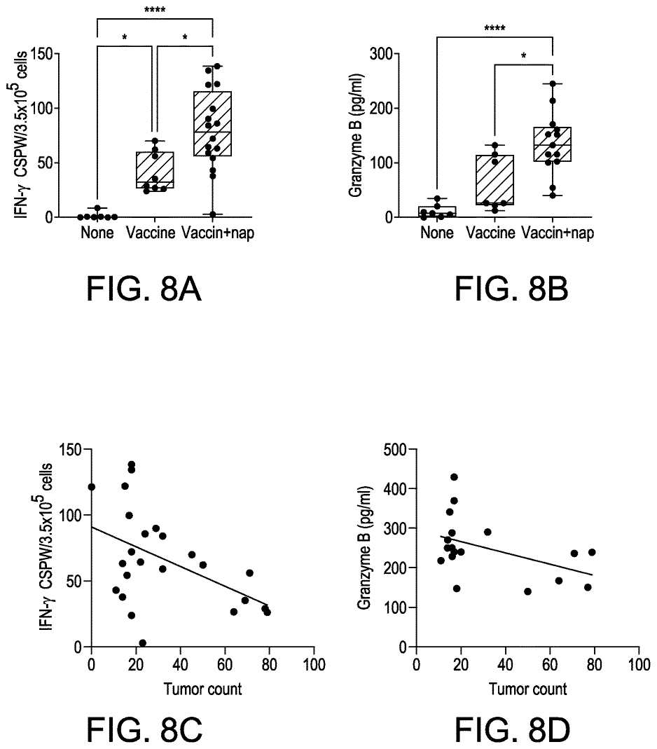

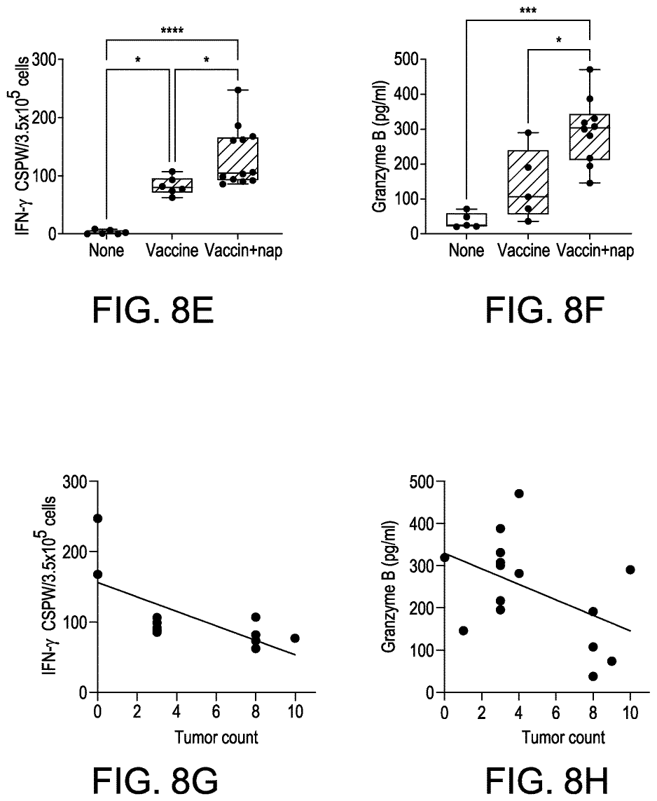

The combination of vaccination with naproxen increased the antigen specific immune response which was associated with tumor protection. Immunization with adjuvant and/or treatment with any regimen of naproxen did not result in increased antigen-specific IFN-γ or Granzyme B. Since there was no significant difference in the immune response generated between naproxen regimens, we did not differentiate between them in our subsequent analyses. As compared to control, immunization alone in ApcMin/+ mice induced a significantly higher magnitude IFN-γ response (41±7 CSPW; p=0.034) which was even higher when mice were also treated with naproxen (mean, 75±11.3 CPSW; p=0.01; FIG. 8A). While vaccine alone did not significantly increase Granzyme B levels, when vaccine was combined with naproxen therapy, Granzyme B levels were significantly increased (mean, 134±16 μg/mL; p=0.01; FIG. 8B). A smaller number of lesions present in the small bowel was positively correlated with either increased IFN-γ CSPW (Pearson correlation coefficient: −0.5004, p=0.0177; FIG. 8C) or Granzyme B levels (FIG. 8D). Similar results were observed in AOM-treated mice. Treatment with naproxen in immunized mice (132±82 CSPW) significantly increased the magnitude of the IFN-γ response as compared to vaccine alone (82±6 CSPW; p=0.04; FIG. 8D). Granzyme B levels were also significantly increased when vaccine was combined with naproxen therapy (mean, 295±29 μg/mL; p=0.01; FIG. 8E). A smaller number of lesions present in the colon was positively correlated with either increased IFN-γ CSPW (Pearson correlation coefficient: −0.6005, p<0.001; FIG. 8F) or Granzyme B levels (FIG. 8G).

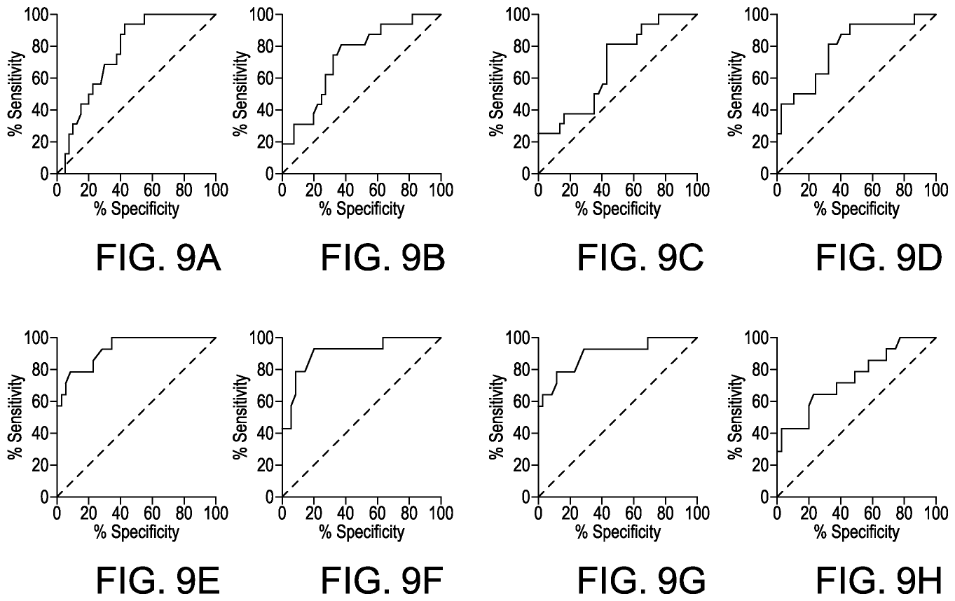

Tumor infiltrating lymphocyte biomarkers of the immune response with the combination of naproxen and vaccine can be identified. Immunophenotyping of the tumor infiltrating lymphocytes was performed in both mouse models, evaluating memory T-cells, T-regulatory cells, Th2, Th1, cytotoxic T-cells and for the presence of PD-1 on T-cells. The percentage of infiltrating myeloid derived suppressor cells (MDSC) was also assessed (Table 3). Four cell types were significantly associated with differentiating the response observed in either monotherapy alone to the response observed after the combination therapy. In the ApcMin/+ mice, the area under the curve (AUC) for CD8+CD3+ T-cells was 76% (p=0.002; FIG. 9A), for Tbet+CD4+ T-cells was 73% (p=0.008; FIG. 9B), for effector memory CD4+ T-cells was 68% (p=0.041; FIG. 9C) and for effector memory CD8+ T-cells was 79% (p<0.001; FIG. 9D). The same cell phenotypes also could differentiate single and combination therapy in the AOM-treated mice (mean AUC 87%, range 75-93%; p<0.001 for all; FIG. 9E-H).

TABLE 5
Mean percent of indicated cell phenotype

| Mouse | | Adjuvant | | | Vaccine | | | p-value | p-value |
|---|---|---|---|---|---|---|---|---|---|
| Model | Cell Phenotype | none | INT | CON | none | INT | CON | INT | CON |
| APC^{Min/+} | % CD4 of CD3+ | 22 | 14 | 30 | 22 | 26 | 19 | ns | ns |
| | % CD8 of CD3+ | 9 | 15 | 35 | 52 | 44 | 49 | 0.00003 | 0.06 |
| | % CM of CD4+ | 11 | 18 | 14 | 21 | 22 | 11 | ns | ns |
| | % EM of CD4+ | 15 | 41 | 60 | 27 | 75 | 53 | 0.02 | ns |
| | % CM of CD8+ | 2 | 2 | 4 | 16 | 22 | 3 | 0.04 | ns |
| | % EM of CD8+ | 3 | 44 | 48 | 38 | 64 | 57 | 0.05 | ns |
| | % Foxp3 of CD4 | 6 | 2 | 4 | 3 | 5 | 3 | 0.04 | ns |
| | % GATA3 of CD4+ | 18 | 4 | 5 | 6 | 8 | 4 | 0.02 | ns |
| | % Tbet of CD3 | 1 | 11 | 9 | 1 | 14 | 11 | ns | ns |
| | % PD1 of CD4+ | 3 | 5 | 3 | 3 | 4 | 4 | ns | ns |
| | % PD1 of CD8+ | 1 | 7 | 7 | 1 | 4 | 4 | 0.0005 | ns |
| | % MDSC | 0 | 4 | 1 | 1 | 1 | 0 | 0.00001 | ns |
| AOM | % CD4 of CD3+ | 62 | 66 | 61 | 69 | 69 | 65 | ns | ns |
| | % CD8 of CD3+ | 5 | 11 | 12 | 12 | 18 | 18 | ns | 0.01 |
| | % CM of CD4+ | 3 | 6 | 6 | 6 | 5 | 5 | ns | ns |
| | % EM of CD4+ | 34 | 48 | 43 | 42 | 68 | 71 | ns | 0.00 |
| | % CM of CD8+ | 6 | 6 | 8 | 8 | 8 | 8 | ns | ns |
| | % EM of CD8+ | 29 | 53 | 52 | 52 | 67 | 66 | ns | 0.04 |
| | % Foxp3 of CD4 | 15 | 5 | 5 | 4 | 2 | 1 | ns | 0.03 |
| | % GATA3 of CD4+ | 10 | 3 | 2 | 3 | 2 | 2 | ns | ns |
| | % Tbet of CD3 | 3 | 9 | 10 | 8 | 17 | 18 | ns | 0.01 |
| | % PD1 of CD4+ | 3 | 4 | 3 | 4 | 3 | 3 | ns | ns |
| | % PD1 of CD8+ | 5 | 4 | 4 | 3 | 4 | 3 | ns | ns |
| | % MDSC | 10 | 6 | 6 | 2 | 1 | 1 | 0.03 | 0.02 |

### REFERENCES

1. Biswas, S., et al., Transforming growth factor beta receptor type II inactivation promotes the establishment and progression of colon cancer. Cancer Res, 2004. 64 (14): p. 4687-92.
2. Cecil, D. L., et al., Immunization against HIF-1alpha Inhibits the Growth of Basal Mammary Tumors and Targets Mammary Stem Cells In Vivo. Clin Cancer Res, 2017. 23 (13): p. 3396-3404.
3. Denise L. Cecil, B. C., Ekram Gad, Michael Gormley, Andrew E. Timms, Lauren Corulli, Rinke Bos, Rajendra N Damle, Manuel A. Sepulveda and Mary L. Disis, Anti-tumor activity of a T-helper 1 multiantigen vaccine in a murine model of prostate cancer Scientific Reports, 2022. in press.
4. Cecil, D. L., et al., COX-2 Inhibitors Decrease Expression of PD-L1 in Colon Tumors and Increase the Influx of Type I Tumor-infiltrating Lymphocytes. Cancer Prev Res (Phila), 2022. 15 (4): p. 225-231.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A nucleic acid molecule comprising a nucleic acid sequence encoding a polypeptide comprising at least three epitopes selected from the group consisting of:
(a) amino acids 132-156 of CDC25B;
(b) amino acids 76-93 and 276-295 of COX2;
(c) amino acids 38-53, 60-82, and 93-117 of HIF-1alpha;
(d) amino acids 93-110 and 137-156 of CDH3;
(e) amino acids 880-895 and 962-976 of EGFR; and
(f) an amino acid sequence having at least 90% identity with any one of the foregoing.

2. The nucleic acid molecule of claim 1, wherein the polypeptide comprises:
(a) amino acids 132-156 of CDC25B;
(b) amino acids 76-93 and 276-295 of COX2;
(c) amino acids 38-53, 60-82, and 93-117 of HIF-1alpha;
(d) amino acids 93-110 and 137-156 of CDH3; and
(e) amino acids 880-895 and 962-976 of EGFR.

3. The nucleic acid molecule of claim 1, wherein the polypeptide comprises:
(a) amino acids 124-164 of CDC25B;
(b) amino acids 72-97 and 274-295 of COX2;
(c) amino acids 30-119 of HIF-1alpha;
(d) amino acids 93-164 of CDH3; and
(e) amino acids 877-901 and 960-980 of EGFR.

4. The nucleic acid molecule of claim 1, wherein the polypeptide comprises at least one of each of (a), (b), (c), (d), and (e), and optionally further comprises a linker sequence disposed between the epitopes.

5. The nucleic acid molecule of claim 4, wherein the linker sequence is GGGGGS (SEQ ID NO: 16) or GPGPG (SEQ ID NO: 17).

6. The nucleic acid molecule of claim 1, wherein the nucleic acid sequence comprises a promoter sequence.

7. The nucleic acid molecule of claim 1, wherein the nucleic acid sequence comprises a CMV enhancer.

8. The nucleic acid molecule of claim 1, wherein the nucleic acid sequence comprises a heterologous sequence.

9. The nucleic acid molecule of claim 8, wherein the heterologous sequence encodes a promoter, a transcriptional start site, a translational start site, a detectable marker, a mRNA processing splice site, a polyadenylation sequence, and/or a regulatory element.

10. A vector comprising the nucleic acid molecule of claim 1, wherein the vector is capable of directing expression of the encoded polypeptide(s).

11. The vector of claim 10, wherein the vector is a DNA plasmid, messenger RNA (mRNA), or viral vector.

12. The vector of claim 11, wherein the viral vector is a lentiviral vector, an adenoviral vector, or a poxviral vector.

13. The vector of claim 12, wherein the vector is mRNA.

14. The vector of claim 10, wherein the vector comprises the nucleotide sequence of SEQ ID NO: 19 or encodes the polypeptide of SEQ ID NO: 18.

15. A polypeptide comprising at least three epitopes selected from the group consisting of:
(a) amino acids 132-156 of CDC25B;
(b) amino acids 76-93 and 276-295 of COX2;
(c) amino acids 38-53, 60-82, and 93-117 of HIF-1alpha;
(d) amino acids 93-110 and 137-156 of CDH3;
(e) amino acids 880-895 and 962-976 of EGFR; and
(f) an amino acid sequence having at least 90% identity with any one of the foregoing.

16. A composition comprising the polypeptide of claim 15, wherein the polypeptide comprises at least one of each of (a), (b), (c), (d), and (e), and optionally further comprises one or more linker sequences disposed between the epitopes.

17. A composition comprising the polypeptide of claim 15, wherein the composition further comprises an adjuvant.

18. A method for eliciting a Type 1 immune response directed at colorectal cancer, for preventing or treating colorectal tumors, and/or for ameliorating the symptoms of colorectal cancer in a subject in need thereof, the method comprising administering to the subject a composition comprising the nucleic acid molecule of claim 1.

19. The method of claim 18, wherein the Type 1 immune response is an IFN-gamma response.

20. The method of claim 18, wherein the subject is human.
